(19) United States
(12) Patent Application Publication
(10) Pub. No.: US 2016/0089551 A1
(43) Pub. Date: Mar. 31, 2016

(54) HIGH-INTENSITY FOCUSED ULTRASOUND THERAPY SYSTEM WITH COOLING

(71) Applicant: KONINKLIJKE PHILIPS N.V., Eindhoven (NL)

(72) Inventors: MIKA PETRI YLIHAUTALA, Eindhoven (NL); TUOMO TAPIO ANTTILA, EINDHOVEN (NL); ANNEMARIA JOHANNA HALKOLA, EINDHOVEN (NL); MATTI OSKARI TILLANDER, Eindhoven (NL); MAX OSKAR KOEHLER, EINDHOVEN (NL)

(21) Appl. No.: 14/890,647

(22) PCT Filed: May 14, 2014
(86) PCT No.: PCT/EP2014/059808

§ 371 Date: Nov. 12, 2015

(30) Foreign Application Priority Data
May 15, 2013 (EP) 13167758.5

(51) Int. Cl. A61N 7/02; A61B 5/055

(52) U.S. Cl. CPC A61N 7/02; A61B 5/055

(57) ABSTRACT

A high-intensity focused ultrasound (HIFU) therapy system comprises an ultrasound transduce to emit a focused ultrasound beam along a beam path. An ultrasound transparent window is positioned in the beam path. A fluid cooling system to provide cooling of an object to which the focused ultrasound beam is directed. The fluid cooling system includes a fluid receptacle mounted adjacent to the ultrasound transparent window and a cooling unit to cool a coolant and pass the coolant trough the fluid receptacle to and from the fluid receptacle. A degassing module and preferably also a filter to remove volatile components from the coolant. Dissolved air or other gases are removed from the coolant, so that the formation of bubbles in the coolant is avoided or at least suppressed.

### FIELD OF THE INVENTION

The invention pertains to a high-intensity focused ultrasound therapy system comprising a fluid cooling system.

### BACKGROUND OF THE INVENTION

Such a high-intensity focused ultrasound therapy system is known from the international application WO2012/052847.

The known high-intensity focused ultrasound therapy system actively adjusts temperature of non-target tissue by actively cooling the non-target tissue. The ultrasound radiation is focused into a target tissue to actively heat the target tissue. A temperature field is monitored in a region encompassing the target tissue and the non-target tissue. The ultrasound focusing is adjusted, by adjusting the location of the focus or adjusting the intensity of the focus on the basis of the monitored temperature field.

The international application WO2012/098482 mentions a HIFU system that is integrated with a magnetic resonance imaging system. However, no mention is made a cooling system for cooling the object to be sonicated. The US-patent application US 2008/0077056 discloses a HIFU system with a fluid circulation system and a degasser. A fluid inlet and outlet are provided in communication with a fluid pathway adjacent to the face of the transducer. The fluid circulation of the known HIFU system functions to chill the rectal wall of the patient.

### SUMMARY OF THE INVENTION

An object of the invention is to provide a high-intensity focused ultrasound (HIFU) therapy system which more accurately avoids unwanted heating of sensitive tissue of the patient to be treated.

This object is achieved by the high-intensity focused ultrasound (HIFU) therapy system according to the invention comprising an ultrasound transducer to emit a focused ultrasound beam along a beam path

an ultrasound transparent window positioned in the beam path and

a fluid cooling system to provide cooling of an object to which the focused ultrasound beam is directed, wherein

the fluid cooling system includes

a fluid receptacle mounted adjacent to the ultrasound transparent window, the ultrasound transparent window is mounted in the support face of the patient carrier onto which the patient to be treated is placed.

a cooling unit to cool a coolant and pass the coolant trough the fluid receptacle and

a degassing module to remove volatile components such as air or gas from the coolant.

An insight of the present invention is that there when the volatile components, such as dissolved air or other gases or air bubbles, are removed from the coolant, the formation of bubbles in the coolant is avoided or at least suppressed. Such bubbles could interfere with the ultrasound beam transmitted through the coolant. Notable, any “particle” that has significantly different acoustic impedance than the coolant itself can interfere with the ultrasound beam transmitted through the coolant transmitted through the coolant. This can be for example, any gas bubble or solid particle. Such bubbles when they occur, may perturb the ultrasound beam and the ultrasound focus due to scattering and reflection of ultrasound radiation emitted from the ultrasound transducer. Furthermore, dissolved air or other gases form microbubbles which may more easily induce cavitation and as consequence rapid localized heating, when exposed to intense ultrasound field. As a consequence bubbles and dissolved air or gases in the coolant can cause uncontrolled heating in tissue, notably in the patient's skin, that should not be heated. By avoiding these bubbles to be formed, such undesired heating of tissue outside a target zone in which ultrasound radiation is to be focused, is effectively avoided. In particular skin burns of the patient to be treated are avoided.

The high-intensity focused ultrasound (HIFU) therapy system of the invention is fitted with the ultrasound transducer which generates the focused ultrasound beam. Notably, a transducer array is employed which has a plurality of transducer elements, usually arranged in a matrix format, but also random spatial arrangements of the transducer elements are possible. The focus is generated and controlled (electronically) by adjusting the phase of the individual transducer elements. Also the ultrasound transducer as whole can be mechanically displaced, translated and rotated to adjust the position of the focus. The focused ultrasound beam is emitted through the ultrasound transparent window. The ultrasound transparent window is for example integrated in the patient carrier, on which the patient to be treated is placed for treatment by focused ultrasound radiation. In particular, the ultrasound transparent window is mounted in the support face of the patient carrier onto which the patient to be treated is placed. The ultrasound transducer is for example mounted in a transducer tank, also mounted in the patient carrier, for example below the support face. The fluid receptacle is mounted on top of the transducer tank, i.e. at the side facing the support face of the patient carrier. The transducer tank is usually filled with a substance that has good ultrasound transmission and which has an ultrasound impedance that is close to or equal to the ultrasound impedance of the patient to be treated; water is a good choice alternatively a liquid that has acoustic impedance close to water can be used. For example many oils are suitable. However, in the magnetic resonance imaging (MRI) guided HIFU systems liquid also should have favorable MRI properties. Water, with high dielectric permittivity, affects the radio frequency transmission field (so called B₁ field) of the MRI scanner by shortening the wavelength, if introduced in larger quantities in the MRI scanner. As a consequence the transmission field may be distorted, especially in the high field strength MRI scanners such as 3T MRI scanners. Therefore suitable oil or other liquid with lower dielectric permittivity than water is often used in the transducer tank of the MRI guide HIFU systems.

During the HIFU therapy patient lies on top of the ultrasound window, either directly or via coupling medium such as gel pad, so that the acoustic energy can be transmitted to the patient. Human thermoregulation system keeps the body core temperature close to 37° C., and the temperature drops through subcutaneous fat and skin layers as well as through the ultrasound contact medium and ultrasound window towards liquid temperature in the ultrasound tank, that is typically at the room temperature initially. However, the temperatures of these layers are not constant because patient is a heat source that heats the contact interfaces and the liquid in the ultrasound tank. Furthermore these interfaces and tank liquid can be heated due to acoustical losses of the different layers during ultrasound sonications, as well as due to electrical losses, for example caused by the transducer. As a consequence tank liquid and interface temperature tend to increase over the course of the therapy, which in turn elevates the temperature of skin and subcutaneous fat, and thus increases the risk of the excessive heating of the tissue resulting in burns in the worst case.

In order to avoid the heating of the ultrasound contact and to cool part of the object, notably the patient to be treated, to which the ultrasound beam is directed when the high-intensity focused ultrasound (HIFU) therapy system is in operation, the fluid cooling system is provided. The fluid cooling system includes a fluid receptacle. The fluid coolant is passed through the fluid receptacle, i.e. the fluid coolant is passed to and from the fluid receptacle so that heat taken up by the coolant is carried-off from the receptacle and coolant at lower temperature is supplied to the receptacle. Thus the receptacle with the coolant passing through takes up heat to remove heat from the patient to be treated. The receptacle with the coolant is mounted on top of the transducer tank, for example, the fluid receptacle is formed as a cooling cavity integrated in the patient support, notably integrated on top of the transducer tank. This construction is both simple and provides a good thermal contact between the coolant and the patient's skin. As the fluid receptacle is mounted adjacent to the ultrasound window, thermal contact will be established between the body of the patient to be treated and the fluid receptacle when the patient to be treated is placed in proper position over the ultrasound window for treatment by irradiation by the ultrasound radiation. In order to carry-off heat from the patient to be treated that is taken up by the coolant in the fluid receptacle, the coolant is passed through the fluid receptacle by way of the cooling unit. The cooling unit includes a heat exchanger to cool coolant that returns from the fluid receptacle. The cooling unit then passes the cooled fluid again to the fluid receptacle. The cooling unit is fitted with a fluid pump to generate a flow of coolant through the fluid receptacle.

In order to avoid formation of bubbles, the degassing module is provided in the fluid cooling system to remove volatile components from the coolant.

These and other aspects of the invention will be further elaborated with reference to the embodiments defined in the dependent Claims.

According to an aspect of the invention, self-closing connections, such as quick coupling hydraulic connectors that have check valves, are employed to connect the fluid channels, e.g. tubes, to the fluid receptacle and to the cooling unit. In this way a closed circuit is arranged formed by the cooling unit, the fluid channels and the fluid receptacle into which hardly or no gas or other volatile components can leak into.

According to a further aspect of the invention, the fluid receptacle, e.g. formed as a cooling cavity is fitted with an air-tight enclosure. This further avoids leakage of volatile components into the coolant. This reduces formation of bubbles in the coolant.

According to another aspect of the invention, a filter is provided in the cooling unit to filter out volatile components. In this way any residual amount of volatile components, such as air or other gases, are filtered out from the coolant. Thus, a further reduction of bubble formation in the coolant is achieved.

According to another aspect of the invention a temperature sensor is provided to measure the temperature on the coolant in the fluid receptacle. The fluid cooling system is controlled on the basis of the measured temperature of the coolant in the fluid receptacle to accurately control the temperature of the patient to avoid unwanted heating and notably avoid skin burns during high-intensity focused ultrasound treatment.

Further, the present invention may be incorporated into a magnetic resonance image guided high-intensity focused ultrasound (HIFU) therapy system. The magnetic resonance guiding is provided by a MR thermographic imaging module that is configured to derive a temperature distribution from acquired magnetic resonance signals. The temperature distribution is for example derived from the phase of the magnetic resonance signals making use of the proton resonance frequency shift with temperature. Alternatively, the temperature distribution is derived from the relaxation rate of the magnetic resonance signals, making use of the temperature dependence of e.g. the decay rate (T₁) of the longitudinal magnetisation or (T₂) the transverse magnetization. On the basis of the temperature images. the effect of the cooling to the patient can be monitored, and the actual near field temperature can be taken into account in the therapy execution.

These and other aspects of the invention will be elucidated with reference to the embodiments described hereinafter and with reference to the accompanying drawing wherein

### BRIEF DESCRIPTION OF THE DRAWINGS

FIG. 1 shows a schematic representation of the high-intensity focused ultrasound (HIFU) therapy system of the invention;

FIG. 2 shows a schematic representation of the fluid cooling system of the high-intensity focused ultrasound (HIFU) therapy system of FIG. 1.

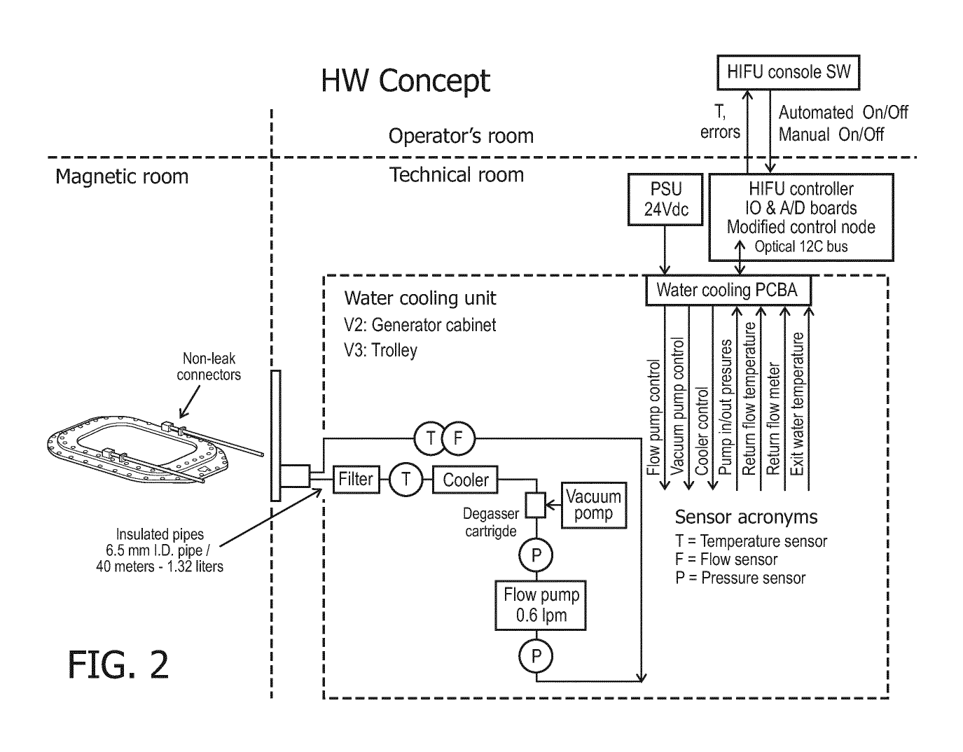

### DETAILED DESCRIPTION OF THE PREFERRED EMBODIMENTS

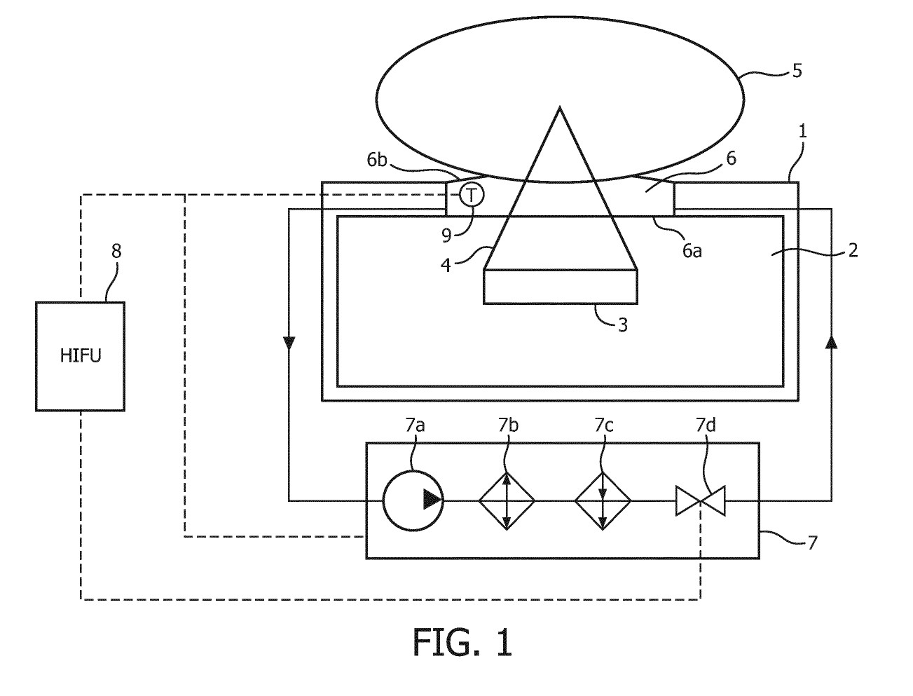

FIG. 1 shows a schematic representation of the high-intensity focused ultrasound (HIFU) therapy system of the invention. The patient support is formed by a HIFU therapy table (1) which contains the transducer tank in the form of a liquid reservoir (2) where the ultrasound transducer (3) is located. The focused ultrasound beam (4) is transmitted to the patient to be treated (5) for therapy purpose (e.g. to ablate tumours). The ultrasound beam is transmitted through fluid receptacle formed as a cavity (6) filled with ultrasound transparent liquid. Typically this liquid could be water but also other liquids with suitable properties could be used. Selection criteria for the liquid include i) ultrasound properties, ii) cooling properties, and in the case of MR guided HIFU iii) MR properties (such as visibility in the MR images). The lower (6a) and upper (6b) surfaces of the cavity (6) are formed by ultrasound “transparent” materials, i.e. materials through which majority of the ultrasound is transmitted through and only minor part of the ultrasound is reflected or absorbed. Such a condition is arranged by using materials which have acoustic impedance close enough to the acoustic impedance of the reservoir liquid at the lower surface and close to acoustic impedance of tissue at the upper surface or by selecting thicknesses of the surface materials so that acoustic matching is sufficient. Furthermore materials should not penetrate air through so that the formation of the bubbles is avoided. Typically materials would be thin plastics with favorable acoustic properties. In addition the surface materials can be flexible so that the shape of the surface adapt to the shape of the patient anatomy located on top of the upper surface (6b). The cavity (6) may be integrated as part of the HIFU table (1) or it can be removable unit that can be positioned on top of the ultrasound window. In order to reduce the skin and subcutaneous fat temperature and to enhance the cooling of these areas, cooled liquid is circulated through the cavity (6) using the cooling unit that contains a cooling and circulating unit (7). Such unit typically contains pump (7a) for making the liquid circulation, cooling unit (7b) to cool the circulated liquid, and temperature regulator (7c) to control the circulated liquid temperature. Instead of controlling only liquid temperature, the system could also regulate liquid flow or mix suitable temperature liquid from two temperature liquid sources. In addition the liquid circulation may be under external control through valve (7d) or other means (such as controlling the pump). This enables, for example, in the MR guided HIFU therapy the HIFU control unit (8) to switch off or reduce the liquid circulation during the MRI imaging (such as MR-thermometry imaging) to avoid possible image artefacts arising from the flowing liquid in the cavity (6) and in the connecting pipes. The valve (7d) can be opened again to enable cooling circulation when MR-imaging is not performed. Set-up may also have temperature sensor (9) to measure the actual liquid temperature at the cavity (6). Further a sensor can be provided both at the outflow as now indicated but also at the inflow in order to gauge the actual temperature at that point. Typically the temperature is slightly elevated on its way to the fluid receptacle that in this example is formed by a cooling cushion, even though the tubing is intended to be as well isolated as possible. The actual inflow temperature is what will decide whether or not patient cooling would be possible, and the difference to the outflow temperature gives an idea of the heat energy conducted away from the patient. This information may be used either by the HIFU therapy control unit or directly by the cooling and circulating unit to adjust the cooling by setting, e.g., the circulated water temperature or circulation speed. In addition the cavity temperature can be used as safety limit to avoid too low temperatures, which might result in tissue damage or other damage to the patient. Cooling of sensitive regions is known per se in the field of high-intensity focused ultrasound treatment of prostate cancer from the paper by Gelet A, Chapelon J Y, Bouvier R, Pangaud C, Lasne Y. Local control of prostate cancer by transrectal high intensity focused ultrasound therapy: preliminary results. in J. Urol. 1999; 161:156-162. Here it is mentioned that a cooling system allows the coupling liquid circulating in the balloon to protect the rectal mucosa by removing the thermal energy released at the balloon-rectum interface on the firing of each shot. Due to cooling the rectal temperature never exceeds 37 C.

One new important technical aspect of the invention is the handling of e.g. air in the cooling circulation. In order to keep the air and other gases out of the circulation system the whole system is made air/gas tight using materials and designs that do not penetrate air and other gases.

Furthermore the dissolved gas content of the coolant liquid is made sufficiently low to avoid risk of cavitation and to remove air bubbles from coolant liquid by running the circulated water is through degasser unit (10). The degasser unit will remove dissolved gas and air bubbles from the coolant liquid that is circulated through the degasser. The degassing unit includes, for example, degasser cartridge (10a), that typically has membrane structure that allows gas to penetrate through the membrane out of the coolant circulation while keeping coolant liquid in the circulation. Dissolved gas is evacuated from the degasser cartridge with the vacuum pump (10b). Other implementations of the degasser can be used instead of the described one as well. In case that the cooling unit needs be possible to disconnect from the cooling cavity, quick coupling hydraulic connectors that have check valves (11) can be used to avoid leakage of air to the circulation system as well as leakage of the liquid out of the circulation system. Furthermore circulation can include air/gas bubble filter (12) to disable bubbles to enter in the ultrasound window region with the circulation flow. Such air filter typically has mechanical mesh structure that stops the air/gas bubbles larger than the mesh pore sizes and traps the air in the air cavity.

Furthermore, the cooling can be integrated in the patient support. This avoids problems for users to assemble detachable cooled contact on top of the original US window membrane so that no air bubbles are trapped in between, so the integrated concept has workflow benefit. The detachable solution would introduce multiple thin plastic layers close to each other along the beam path, that increase the ultrasound reflections and thus may harm the ultrasound transducer. In the case of the integrated solution less reflected surfaces are required. In addition integrating the cooling unit as a separate intermediate cavity between the ultrasound tank and the patient has advantages that it reduces the volume of liquid that needs to be cooled and thus makes the initial cooling period to reach the target temperature faster. Minimizing the size of the cooling volume, when water is used as cooling agent, is also important in the MRI environment, because any larger volume of water starts to affect the RF transmit field (so called B1 field) homogeneity especially if higher field strength like 3T are used. Integrated solution has also the benefit that it enables the transducer to be moved independent of the cooling, unlike in the transrectal prostate HIFU solutions where the cooling is integrated with the transducer.

Yet another aspect that is important for utilizing the cooled contact is the possibility to measure absolute temperature of the fat to quantify the benefit of the cooling. In our implementation this will be done with the (apparent) T₂ based fat temperature mapping protocol known per se form the international application WO2012/029006. FIG. 3 below illustrates the benefit of this method over the conventional proton resonance frequency shift method (PRFS). PRFS method measures only the temperature change and the starting temperature is “guessed” to be for example the body temperature. Longer duration temperature evolution follow-up is not typically feasible with the PRFS because the method is based on the signal phase measurement and phase is very sensitive to many kinds of errors (system drifts, patient motion, . . . ). The T₂ based method, however, determines the fat temperature from the apparent T₂ value determined form the magnitude images made with two different echo times. The absolute temperature is calculated based on the calibrated apparent T₂ vs. temperature behavior. This enables absolute temperature values to be calculated and temperature changes to be monitored over the whole treatment duration (typically few hours).

1. A high-intensity focused ultrasound (HIFU) therapy system comprising:
an ultrasound transducer configured
to emit a focused ultrasound beam along a beam path;
an ultrasound transparent window positioned in the beam path; and
a fluid cooling system to provide cooling of an object to which the focused ultrasound beam is directed, wherein
the fluid cooling system includes:
a fluid receptacle mounted adjacent to the ultrasound transparent window;
a cooling unit to cool a coolant and pass the coolant through the fluid receptacle; and
a degassing module to remove volatile components such as air or gas from the coolant;
and a patient support wherein the fluid receptacle is formed as a cooling cavity integrated in the patient support at its side for receiving the patient.

2. The high-intensity focused ultrasound (HIFU) therapy system as set forth in claim 1, wherein the cooling unit is provided with a filter to filter volatile components, such as air or gas, from the coolant.

3. The high-intensity focused ultrasound (HIFU) therapy system as set forth in claim 1, wherein the fluid receptacle has an air-tight enclosure.

4. The high-intensity focused ultrasound (HIFU) therapy system as set forth in claim 1 further including one or more fluid channels to supply and receive the coolant which are connected to the cooling unit and the fluid receptacle by way of self-closing connections.

5. The high-intensity focused ultrasound (HIFU) therapy system as set forth in claim 1, further including a temperature sensor to measure the temperature on the coolant in the fluid receptacle.

6. An MRI image guided high-intensity focused ultrasound therapy system comprising:
an MRI module having an examination region and configured to acquire temperature sensitive magnetic resonance signals from the examination region and to reconstruct a spatial temperature distribution from the magnetic resonance signals and
a high-intensity focused ultrasound system as set forth in claim 1, configured so that the beam path passes at least in part through the examination region and wherein
the temperature sensitivity of the magnetic resonance signals is based on the temperature dependence of the transverse relaxation time (T₂) of the part of the object to which the focused ultrasound beam is directed.
